(19) United States
(12) Patent Application Publication
(10) Pub. No.: US 2014/0180117 A1
(43) Pub. Date: Jun. 26, 2014

(54) Preparation and Application of a Piezoelectric Film for an Ultrasound Transducer

(71) Applicant: Volcano Corporation, San Diego, CA (US)

(72) Inventor: Dylan Van Hoven, Oceanside, CA (US)

(73) Assignee: Volcano Corporation, San Diego, CA (US)

(21) Appl. No.: 14/103,214

(22) Filed: Dec. 11, 2013

Related U.S. Application Data
(60) Provisional application No. 61/745,091, filed on Dec. 21, 2012.

(51) Int. Cl. A61B 8/00; G10K 11/18

(52) U.S. Cl. CPC A61B 8/4483; G10K 11/18
USPC 600/462; 367/140; 427/100

(57) ABSTRACT

The present disclosure involves a method of fabricating an ultrasound transducer. A piezoelectric polymer is mixed into a solution containing a first chemical and a second chemical to form a viscous film. In some embodiments, the first chemical includes methyl ethyl ketone (MEK), and the second chemical includes dimethylacetamide (DMA). In other embodiments, the first chemical includes cyclohexanone, and the second chemical includes dimethyl sulfoxide (DMSO). The film is coated onto a wafer and then flashed off during the coating. Thereafter, the film is baked. The second chemical is removed during the baking. Thereafter, the film is annealed. In some embodiments, the annealing is performed using an annealing temperature in a range from about 135 degrees Celsius to about 145 degrees Celsius and an annealing duration in a range from about 17 hours to about 19 hours. The film has a β phase crystallinity greater than 50% after the annealing.

### CROSS-REFERENCE TO RELATED APPLICATIONS

This application claims priority to Provisional Patent Application No. 61/745,091, filed Dec. 21, 2012, and entitled “PREPARATION AND APPLICATION OF A PIEZOELECTRIC FILM FOR AN ULTRASOUND TRANSDUCER,” the disclosure of which is hereby incorporated by reference in its entirety.

### TECHNICAL FIELD

The present disclosure relates generally to intravascular ultrasound (IVUS) imaging, and in particular, to an IVUS ultrasound transducer, such as a piezoelectric micromachined ultrasound transducer (PMUT), used for IVUS imaging.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

IVUS catheters typically employ one or more transducers to transmit ultrasound signals and receive reflected ultrasound signals. However, conventional transducers may still have issues related to fragility, bulky size, inability to focus the ultrasounds waves, poor β phase crystallinity, manufacturing difficulties, etc. Some existing transducers may have acceptable performance in some of the areas above, but may suffer drawbacks in some of the other areas.

Therefore, while conventional transducers are generally adequate for their intended purposes, they have not been entirely satisfactory in every aspect.

### SUMMARY

Ultrasound transducers are used in intravascular ultrasound (IVUS) imaging to assess medical conditions inside a human body. Conventional transducers may have issues related to fragility, bulky size, inability to focus the ultrasounds waves, poor β phase crystallinity, manufacturing difficulties, etc. To overcome these issues, the present disclosure mixes a piezoelectric polymer into a solution containing a first chemical and a second chemical to form a viscous film. The viscous film is then coated onto a wafer, for example in a spin-coating process. The first chemical is substantially flashed off as the viscous film is coated. The viscous film is then baked, during which the second chemical is substantially removed. The viscous film is then annealed to ensure a high β phase crystallinity.

The present disclosure provides various embodiments of an ultrasound transducer for use in intravascular ultrasound (IVUS) imaging. An exemplary ultrasound transducer includes a substrate. An opening is formed in the substrate. A first metal layer is formed over the opening. An adhesion-promoting layer is formed over the first metal layer. A piezoelectric layer is formed over the adhesion-promoting layer. The piezoelectric layer is substantially thicker than the adhesion-promoting layer. In some embodiments, the adhesion-promoting layer and the piezoelectric layer may have substantially similar material compositions. A second metal layer is formed over the piezoelectric layer. The first metal layer, the adhesion-promoting layer, the piezoelectric layer, and the second metal layer are each a part of a transducer membrane of the micromachined ultrasonic transducer. In some embodiments, the opening is filled with a backing material.

The present disclosure also provides a method of fabricating an ultrasound transducer. The method includes mixing a piezoelectric polymer into a solution containing a first chemical and a second chemical to form a viscous film. In some embodiments, the first chemical includes methyl ethyl ketone (MEK), and the second chemical includes dimethylacetamide (DMA). In some other embodiments, the first chemical includes cyclohexanone, and the second chemical includes dimethyl sulfoxide (DMSO). The method includes coating the viscous film onto a wafer. The first chemical is substantially flashed off during the coating. Thereafter, the film undergoes a baking process. The second chemical is substantially removed during the baking process. Thereafter, the film is annealed. The film has a β phase crystallinity greater than 60% after the annealing. In some embodiments, before the coating: an adhesion-promoting layer is applied over the wafer and baked on the wafer. The adhesion-promoting layer is substantially thinner than the film. The film is coated on the adhesion-promoting layer. In some embodiments, the adhesion-promoting layer has a substantially similar material composition as the film.

The present disclosure further provides an ultrasound system. The system includes an imaging component that includes a flexible elongate member and a piezoelectric micromachined ultrasound transducer (PMUT) coupled to a distal end of the elongate member. The PMUT includes: a substrate having a front surface and a back surface opposite the first surface. A well is located in the substrate. The well extends from the back surface of the substrate to, but not beyond, the front surface of the substrate. A dielectric support layer is formed over the well and over the front surface of the substrate. A portion of the dielectric layer formed over the well has an arcuate shape. A transducer membrane is formed conformally over the dielectric support layer. The transducer member includes a piezoelectric element disposed between a first conductive element and a second conductive element. The system includes an interface module configured to engage with a proximal end of the elongate member. The system also includes an intravascular ultrasound processing component in communication with the interface module.

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIGS. 2-3 and 5-10 are diagrammatic cross-sectional side views of an ultrasound transducer at different stages of fabrication according to various aspects of the present disclosure.

FIG. 4 is a flowchart illustrating a method of forming a piezoelectric film for the ultrasonic transducer according to various aspects of the present disclosure.

FIG. 11 is a method for fabricating an ultrasound transducer according to various aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, the present disclosure provides an ultrasound imaging system described in terms of cardiovascular imaging, however, it is understood that such description is not intended to be limited to this application. In some embodiments, the ultrasound imaging system includes an intravascular imaging system. The imaging system is equally well suited to any application requiring imaging within a small cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

There are primarily two types of catheters in common use today: solid-state and rotational. An exemplary solid-state catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the imaging system with a simple electrical cable and a standard detachable electrical connector.

An exemplary rotational catheter includes a single transducer located at a tip of a flexible driveshaft that spins inside a sheath inserted into the vessel of interest. The transducer is typically oriented such that the ultrasound signals propagate generally perpendicular to an axis of the catheter. In the typical rotational catheter, a fluid-filled (e.g., saline-filled) sheath protects the vessel tissue from the spinning transducer and driveshaft while permitting ultrasound signals to freely propagate from the transducer into the tissue and back. As the driveshaft rotates (for example, at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the driveshaft. The same transducer then listens for returning ultrasound signals reflected from various tissue structures, and the imaging system assembles a two dimensional image of the vessel cross-section from a sequence of several hundred of these ultrasound pulse/echo acquisition sequences occurring during a single revolution of the transducer.

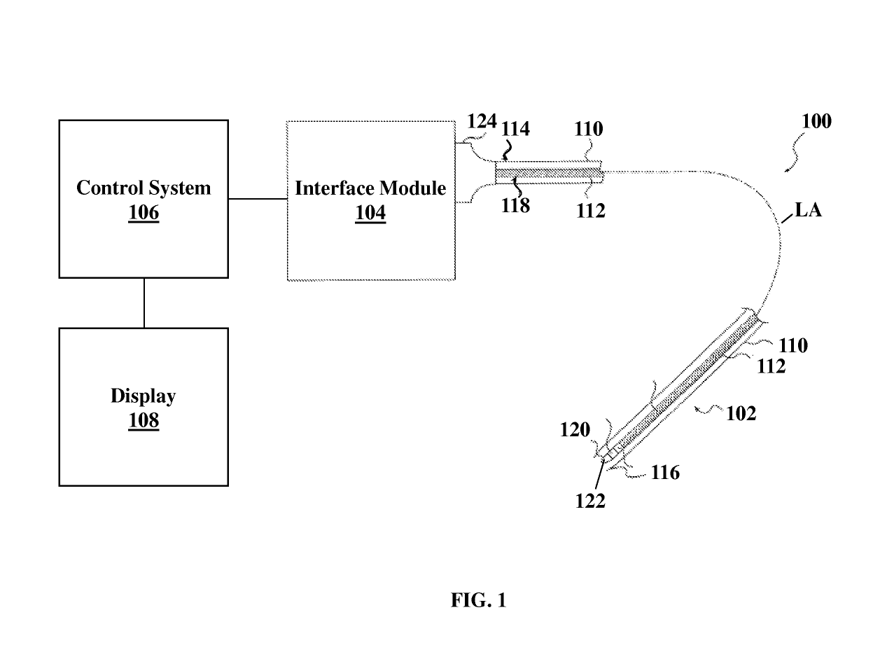

FIG. 1 is a schematic illustration of an ultrasound imaging system 100 according to various aspects of the present disclosure. In some embodiments, the ultrasound imaging system 100 includes an intravascular ultrasound imaging system (IVUS). The IVUS imaging system 100 includes an IVUS catheter 102 coupled by a patient interface module (PIM) 104 to an IVUS control system 106. The control system 106 is coupled to a monitor 108 that displays an IVUS image (such as an image generated by the IVUS system 100).

In some embodiments, the IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 includes an elongated, flexible catheter sheath 110 (having a proximal end portion 114 and a distal end portion 116) shaped and configured for insertion into a lumen of a blood vessel (not shown). A longitudinal axis LA of the catheter 102 extends between the proximal end portion 114 and the distal end portion 116. The catheter 102 is flexible such that it can adapt to the curvature of the blood vessel during use. In that regard, the curved configuration illustrated in FIG. 1 is for exemplary purposes and in no way limits the manner in which the catheter 102 may curve in other embodiments. Generally, the catheter 102 may be configured to take on any desired straight or arcuate profile when in use.

A rotating imaging core 112 extends within the sheath 110. The imaging core 112 has a proximal end portion 118 disposed within the proximal end portion 114 of the sheath 110 and a distal end portion 120 disposed within the distal end portion 116 of the sheath 110. The distal end portion 116 of the sheath 110 and the distal end portion 120 of the imaging core 112 are inserted into the vessel of interest during operation of the IVUS imaging system 100. The usable length of the catheter 102 (for example, the portion that can be inserted into a patient, specifically the vessel of interest) can be any suitable length and can be varied depending upon the application. The proximal end portion 114 of the sheath 110 and the proximal end portion 118 of the imaging core 112 are connected to the interface module 104. The proximal end portions 114, 118 are fitted with a catheter hub 124 that is removably connected to the interface module 104. The catheter hub 124 facilitates and supports a rotational interface that provides electrical and mechanical coupling between the catheter 102 and the interface module 104.

The distal end portion 120 of the imaging core 112 includes a transducer assembly 122. The transducer assembly 122 is configured to be rotated (either by use of a motor or other rotary device or manually by hand) to obtain images of the vessel. The transducer assembly 122 can be of any suitable type for visualizing a vessel and, in particular, a stenosis in a vessel. In the depicted embodiment, the transducer assembly 122 includes a piezoelectric micromachined ultrasonic transducer (“PMUT”) transducer and associated circuitry, such as an application-specific integrated circuit (ASIC). An exemplary PMUT used in IVUS catheters may include a polymer piezoelectric membrane, such as that disclosed in U.S. Pat. No. 6,641,540, hereby incorporated by reference in its entirety. The PMUT transducer can provide greater than 100% bandwidth for optimum resolution in a radial direction, and a spherically-focused aperture for optimum azimuthal and elevation resolution.

The transducer assembly 122 may also include a housing having the PMUT transducer and associated circuitry disposed therein, where the housing has an opening that ultrasound signals generated by the PMUT transducer travel through. Alternatively, the transducer assembly 122 includes a capacitive micromachined ultrasonic transducer (“CMUT”). In yet another alternative embodiment, the transducer assembly 122 includes an ultrasound transducer array (for example, arrays having 16, 32, 64, or 128 elements are utilized in some embodiments).

The rotation of the imaging core 112 within the sheath 110 is controlled by the interface module 104, which provides user interface controls that can be manipulated by a user. The interface module 104 can receive, analyze, and/or display information received through the imaging core 112. It will be appreciated that any suitable functionality, controls, information processing and analysis, and display can be incorporated into the interface module 104. In an example, the interface module 104 receives data corresponding to ultrasound signals (echoes) detected by the imaging core 112 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 122.

In some embodiments, wires associated with the IVUS imaging system 100 extend from the control system 106 to the interface module 104 such that signals from the control system 106 can be communicated to the interface module 104 and/or vice versa. In some embodiments, the control system 106 communicates wirelessly with the interface module 104. Similarly, it is understood that, in some embodiments, wires associated with the IVUS imaging system 100 extend from the control system 106 to the monitor 108 such that signals from the control system 106 can be communicated to the monitor 108 and/or vice versa. In some embodiments, the control system 106 communicates wirelessly with the monitor 108.

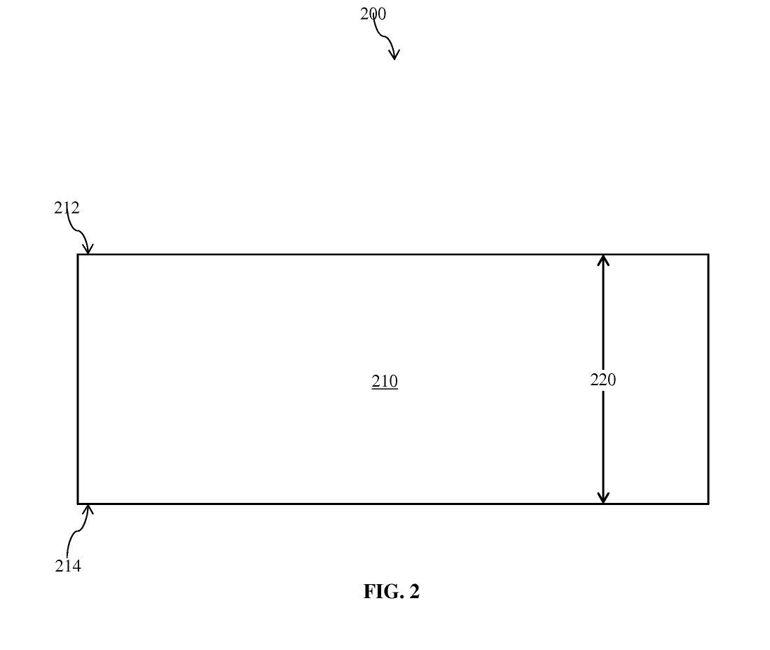

FIGS. 2-3 and 5-10 are diagrammatic fragmentary cross-sectional side views of an ultrasound transducer 200 at different stages of fabrication in accordance with various aspects of the present disclosure. FIGS. 2-3 and 5-10 have been simplified for the sake of clarity to better understand the inventive concepts of the present disclosure.

The ultrasound transducer 200 can be included in the IVUS imaging system 100 of FIG. 1, for example in the transducer assembly 122. The ultrasonic transducer 200 has a small size and achieves a high resolution, so that it is well suited for intravascular imaging. In some embodiments, the ultrasonic transducer 200 has a size on the order of tens or hundreds of microns, can operate in a frequency range between about 1 mega-Hertz (MHz) to about 135 MHz, and can provide sub 50 micron resolution while providing depth penetration of at least 10 millimeters (mm). Furthermore, the ultrasonic transducer 200 is also shaped in a manner to allow a developer to define a target focus area based on a deflection depth of a transducer aperture, thereby generating an image that is useful for defining vessel morphology, beyond the surface characteristics. The various aspects of the ultrasound transducer 200 and its fabrication are discussed in greater detail below.

In the depicted embodiment, the ultrasound transducer 200 is a piezoelectric micromachined ultrasound transducer (PMUT). In other embodiments, the transducer 200 may include an alternative type of transducer. Additional features can be added in the ultrasound transducer 200, and some of the features described below can be replaced or eliminated for additional embodiments of the ultrasound transducer 200.

Referring now to FIG. 2, the transducer 200 includes a substrate 210. The substrate 210 has a surface 212 and a surface 214 that is opposite the surface 212. The surface 212 may also be referred to as a front surface or a front side, and the surface 214 may also be referred to as a back surface or a back side. In the depicted embodiment, the substrate 210 is a silicon microelectromechanical system (MEMS) substrate. The substrate 210 includes another suitable material depending on design requirements of the PMUT transducer 200 in alternative embodiments. In the illustrated embodiments, the substrate 210 is a “lightly-doped silicon substrate.” In other words, the substrate 210 comes from a silicon wafer that is lightly doped with a dopant and as a result has a resistivity in a range from about 1 ohms/cm to about 1000 ohms/cm. One benefit of the “lightly-doped silicon substrate” 210 is that it is relatively inexpensive, for example in comparison with pure silicon or undoped silicon substrates. Of course, it is understood that in alternative embodiments where cost is not as important of a concern, pure silicon or undoped silicon substrates may also be used.

The substrate 210 may also include various layers that are not separately depicted and that can combine to form electronic circuitry, which may include various microelectronic elements. These microelectronic elements may include: transistors (for example, metal oxide semiconductor field effect transistors (MOSFET), complementary metal oxide semiconductor (CMOS) transistors, bipolar junction transistors (BJT), high voltage transistors, high frequency transistors, p-channel and/or n-channel field effect transistors (PFETs/NFETs)); resistors; diodes; capacitors; inductors; fuses; and/or other suitable elements. The various layers may include high-k dielectric layers, gate layers, hard mask layers, interfacial layers, capping layers, diffusion/barrier layers, dielectric layers, conductive layers, other suitable layers, or combinations thereof. The microelectronic elements could be interconnected to one another to form a portion of an integrated circuit, such as a logic device, memory device (for example, a static random access memory (SRAM)), radio frequency (RF) device, input/output (I/O) device, system-on-chip (SoC) device, other suitable types of devices, or combinations thereof.

A thickness 220 of the substrate 210 is measured between the surface 212 and the surface 214. In some embodiments, the thickness 220 is in a range from about 100 microns (um) to about 600 um.

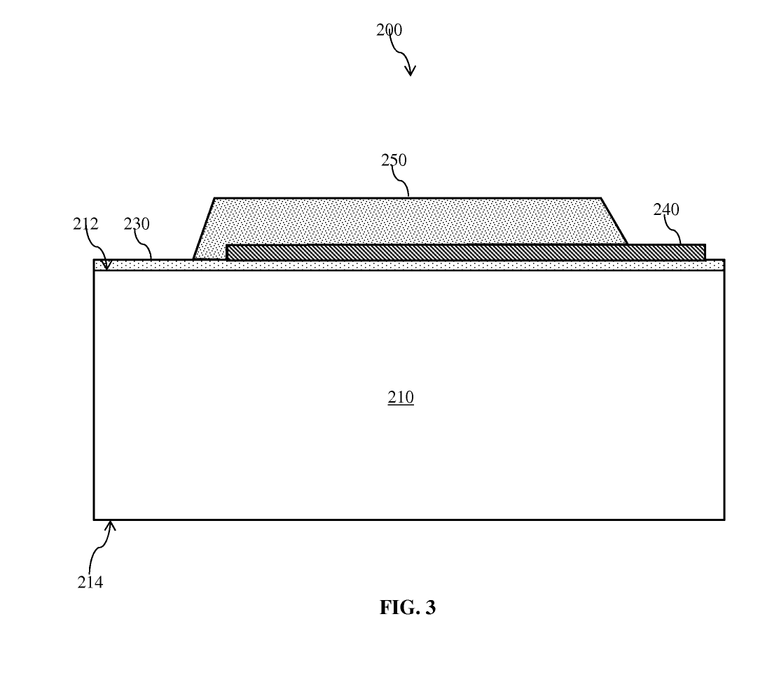

Referring now to FIG. 3, a dielectric layer 230 is formed over the surface 212 of the substrate 210. The dielectric layer 230 may be formed by a suitable deposition process known in the art, such as chemical vapor deposition (CVD), physical vapor deposition (PVD), atomic layer deposition (ALD), or combinations thereof. The dielectric layer 230 may contain an oxide material or a nitride material, for example silicon oxide, silicon nitride, or silicon oxynitride. The dielectric layer 230 provides a support surface for the layers to be formed thereon. The dielectric layer 230 also provides electrical insulation. In more detail, the substrate 210 in the illustrated embodiments is a “lightly-doped silicon substrate” that is relatively conductive, as discussed above. This relatively high conductivity of the substrate 210 may pose a problem when the transducer 200 is pulsed with a relatively high voltage, for example with an excitation voltage of about 60 volts to about 200 volts DC. This means that it is undesirable for a bottom electrode (discussed below in more detail) of the transducer 200 to come into direct contact with the silicon substrate 210. According to the various aspects of the present disclosure, the dielectric layer 230 helps insulate the bottom electrode of the transducer 230 from the relatively conductive surface of the silicon substrate 210.

A conductive layer 240 is then formed over the dielectric layer 230. The conductive layer 240 may be formed by a suitable deposition process such as CVD, PVD, ALD, etc. In the illustrated embodiment, the conductive layer 240 includes a metal material. The conductive layer 240 is patterned using techniques in a photolithography process. Unwanted portions of the conductive layer 240 are removed as a part of the photolithography process. For reasons of simplicity, FIG. 3 only illustrates the conductive layer 240 after it has been patterned.

A piezoelectric film 250 is then formed over the dielectric layer 230 and the conductive layer 240. In various embodiments, the piezoelectric film 250 may include piezoelectric materials such as polyvinylidene fluoride (PVDF) or its co-polymers, polyvinylidene fluoride-trifluoroethylene (PVDF-TrFE), or polyvinylidene fluoride-tetrafluoroethylene (PVDF-TFE). Alternatively, polymers such as PVDF-CTFE or PVDF-CFE may be used. In the illustrated embodiment, the piezoelectric material used in the piezoelectric film 250 contains PVDF-TrFE.

One consideration for a piezoelectric material such as the PVDF-TrFE material of the piezoelectric film 250 is β phase crystallinity. β phase crystallinity is important when using PVDF-TrFE in piezoelectric applications, as the β phase crystallinity is a crystalline phase that is capable of retaining permanent polarization, which is needed for a semi crystalline polymer to become piezoelectric. Some commercially available PVDF-TrFE materials are capable of achieving adequate β phase crystallinity levels. However, existing commercially available PVDF-TrFE materials are typically formed by melt processes and are fragile in nature. Melt processes generally yield films that are difficult to incorporate into MEMS devices. For example, the fragility of the existing PVDF-TrFE materials as a result of the melt processes, and the sheer scale of the coronary anatomy, make this melt-processed PVDF-TrFE materials poor choice for the piezoelectric film in an IVUS transducer.

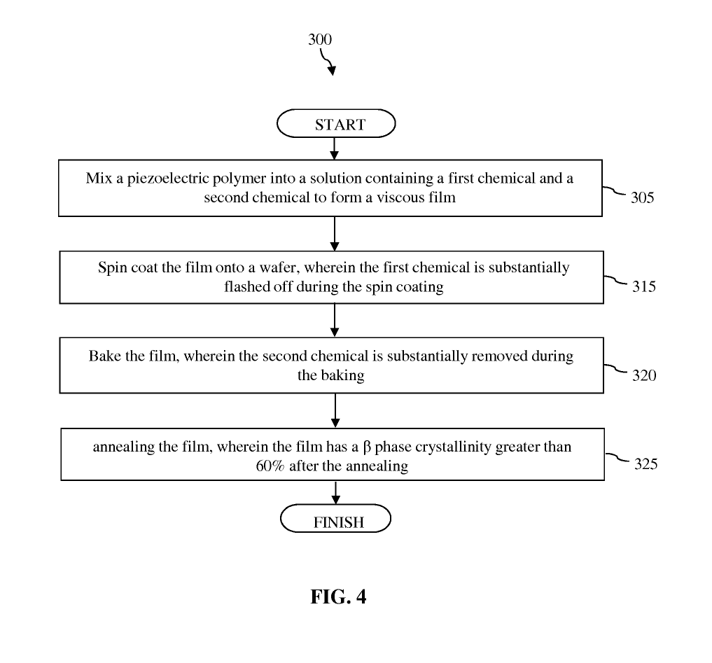

Unlike conventional piezoelectric films formed by melt processes, the piezoelectric film 250 of the present disclosure is formed at least in part by a spin casting process (also referred to as a spin coating process). Achieving a high level of β phase crystallinity has been a challenge for spin casting processes. Therefore, discussed below is a method of forming a high β phase crystallinity piezoelectric film 250 in a spin casting process. In more detail, one aspect of the present disclosure involves a method used to put a piezoelectric polymer such as PVDF-TrFe into a solution, spin cast it onto a wafer (such as a silicon wafer), and anneal it so that it exhibits the level of β phase crystallinity needed for a piezoelectric IVUS transducer. The detailed steps of such method are discussed below with reference to FIG. 4.

Referring to FIG. 4, a simplified flow chart of a method 300 of forming a piezoelectric film is illustrated. The method 300 includes a step 305, in which a piezoelectric polymer is mixed into a solution containing a first chemical (also referred to as a first solvent) and a second chemical (also referred to as a second solvent) to form a viscous film. The piezoelectric polymer may include PVDF-TrFE in the present embodiments but may include PVDF, PVDF-TFE, PVDF-CTFE, PVDF-CFE, or combinations thereof in other embodiments. In yet other alternatively embodiments, the piezoelectric polymer may include piezoelectric materials such as ceramics including ZnO, AlN, LiNbO₄, lead antimony stannate, lead magnesium tantalate, lead nickel tantalate, titanates, tungstates, zirconates, niobates of lead, barium, bismuth, or strontium (for example, lead zirconate titanate (Pb(ZrₓTi₁₋ₓ)O₃ (PZT)), lead lanthanum zirconate titanate (PLZT), lead niobium zirconate titanate (PNZT), BaTiO₃, SrTiO₃, lead magnesium niobate, lead nickel niobate, lead manganese niobate, lead zinc niobate, lead titanate), or combinations thereof.

In some embodiments, the first chemical includes methyl ethyl ketone (MEK), and the second chemical includes dimethylacetamide (DMA). In some other embodiments, the first chemical includes cyclohexanone, and the second chemical includes dimethyl sulfoxide (DMSO). To achieve a desired viscosity in a range from about 575 centipoise (cP) to about 625 cP, a mixing ratio by weight of the piezoelectric polymer, the first chemical, and the second chemical is carefully adjusted. In certain embodiments, such mixing ratio is adjusted such that the piezoelectric polymer varies within a range from about 2 to 3, the first chemical varies within a range from about 6 to 8, and the second chemical varies within a range from about 2 to 4. In that case, the mixing ratio may be expressed as (2˜3):(6˜8):(2˜4). In some other embodiments, the mixing ratio is adjusted such that the piezoelectric polymer varies within a range from about 2.5 to 2.8, the first chemical varies within a range from about 6.5 to 7.5, and the second chemical varies within a range from about 2.5 to 3.5. In that case, the mixing ratio may be expressed as (2.5˜2.8):(6.5˜7.5):(2.5˜3.5). In yet other embodiments, the mixing ratio of the piezoelectric polymer, the first chemical, and the second chemical by weight is about 2.66:7:3.

The viscosity range specified above (between about 575 to 625 cP) facilitates the spin casting of film having a thickness range between about 8 um to about 10 um to a wafer at about 800 revolutions-per-minute (rpm) to about 1000 rpm. A film in this thickness range, for example with a thickness close to a 9 um may be needed to achieve a center frequency of about 40 mega-Hertz (mHz) for the ultrasonic transducer.

Stating the above differently, to achieve a particular center frequency range (e.g., about 40 mHz) for the ultrasonic transducer of the present disclosure, a piezoelectric film with a certain thickness (e.g., about 9 um) needs to be spin cast onto a wafer. To make sure the piezoelectric film can be spin cast onto the wafer, the piezoelectric material needs to have a certain viscosity range (e.g., between about 200 cP to about 1500 cP). In order to achieve this certain viscosity range, the various chemical components used to form the piezoelectric material are configured to have a target mixing ratio (e.g., 2.66:7:3 by weight for PVDF-TrFE:MEK:DMA). However, it is understood that other embodiments may employ different center frequencies for the ultrasonic transducer, which according to the above discussions would lead to a different mixing ratio for the piezoelectric polymer and the other mixing chemicals.

The method 300 includes a step 315, in which the viscous film is spin coated (or spin cast) onto a wafer. The first chemical is substantially flashed off during the spin coating process. In more detail, a wafer on which the piezoelectric material is spin coated over is about a 6-inch silicon wafer in the embodiments of the present disclosure. This is a relatively large area for the piezoelectric material to be evenly spin coated over. The need for the even spin coating of the piezoelectric material over a large wafer surface is one of the reasons for needing the two chemicals or solvents discussed above—MEK and DMA in some embodiments, and cyclohexanone and DMSO in other embodiments.

The reasons for having two solvents are now discussed below using the solvents MEK and DMA as the example first and second solvents. MEK has a vapor pressure of about 71 millimeter of mercury (mmHg) at 20 degrees Celsius. If only MEK was used as a solvent, it would flash off by the time the solvent made its way to the perimeter of the wafer. On the other hand, DMA has a lower vapor pressure of about 2 mmHg at about 25 degrees Celsius. This low vapor pressure of the DMA allows it to not be flashed off until oven baked. However, if only DMA was used, it may not be able to allow the PVDF-TrFE to be spin coated sufficiently evenly.

By using both solvents MEK and DMA, the MEK is allowed to flash off during the spin coating, while the DMA remains to carry the PVDF-TrFE out to the edge of the wafer. When spin coating is finished, most of the solvent mix is evaporated (i.e., MEK has been evaporated during spin coating), leaving a film that is partially set up. The remainder of the solvent (i.e., mostly DMA now) is then baked off in an oven.

In addition to its low vapor pressure, DMA was selected because it has relatively high solids solubility for PVDF-TrFE (i.e., the piezoelectric polymer). It is possible to make solutions of DMA and PVDF-TrFE that are up to about 20% to about 22% PVDF-TrFE. These solutions yield high viscosities of upwards of about 1500 cP. This is beneficial because MEK alone only dissolves enough PVDF-TrFE to yield a solution with a maximum viscosity of about 250 cP, which is not high enough to produce about a 9 um thick film via spin casting. DMSO also dissolves large amounts of PVDF-TrFE and produces solutions with high viscosities, however. This is one of the reasons why DMA was chosen as the second solvent in the solution discussed above. And as discussed above, in alternative embodiments, cyclohexanone and DMSO may be used to substitute MEK and DMO as the first and second chemicals, respectively.

The method 300 includes a step 320, in which the film is baked after it has been spin coated onto the wafer. The second chemical is substantially removed during the baking. As discussed above, the second chemical (e.g., DMA or DMSO) is baked off during the baking process, which is performed after the film has been substantially evenly spin coated onto the wafer.

The method 300 includes a step 325, in which the film is annealed to create a β phase crystallinity needed for an IVUS transducer. In some embodiments, Differential Scanning calorimetry (DSC) analysis of 80:20 PVDF-TrFE was performed to determine the target annealing temperature. According to the experimental results of the DSC analysis, complete crystallite melting of PVDF-TrFE occurs at approximately 145 degrees Celsius. This information is used to perform a Design of Experiments (DOE) evaluating crystallite formation over time at various temperatures around 145 degrees Celsius. Based on the above, the spin cast PVDF-TrFE films may be annealed at a target annealing temperature between about 135 degrees Celsius and 145 degrees Celsius for a target annealing duration between about 17 hours and 19 hours. In the present embodiments, the annealing temperature is about 140 degrees Celsius, and the annealing duration is about 18 hours. This produces a piezoelectric film having a β phase crystallinity greater than 50% after the annealing. In some embodiments, a piezoelectric film having a β phase crystallinity greater than 60% can be produced after the annealing. For example, a piezoelectric film having a β phase crystallinity of about 63% may be achieved. In comparison, commercially available melt processed PVDF-TrFE films typically exhibit a β phase crystallinity less than about 60%. Therefore, according to the various aspects of the present disclosure, a high quality piezoelectric film with a high β phase crystallinity can be formed using a spin coating process, rather than melt processes.

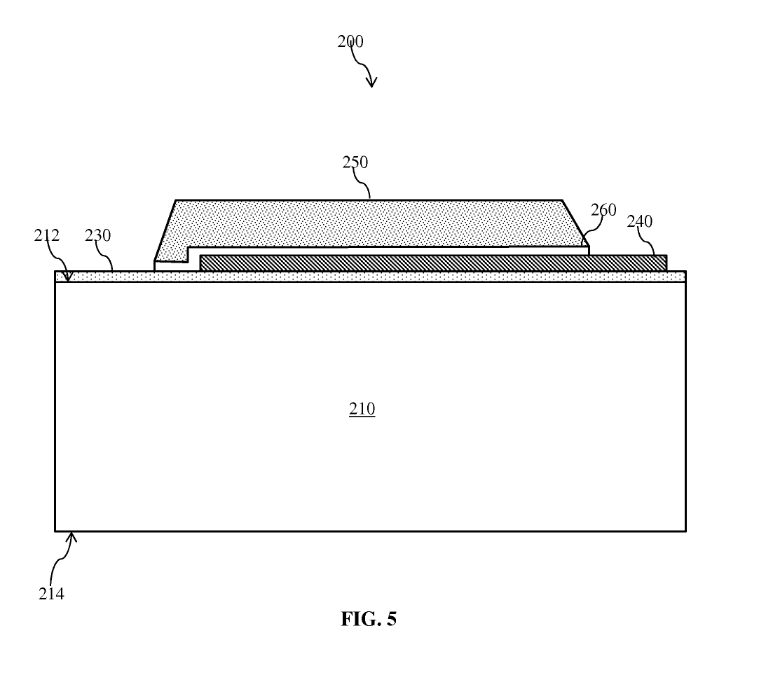

In some embodiments, to further ensure the success of the spin coating process, an adhesion promoter or primer layer can be added over the conductive layer 240 before the piezoelectric film is formed. This is illustrated in FIG. 5, where an adhesion-promoting layer 260 is shown as a part of the transducer 200. The adhesion-promoting layer 260 is formed between the conductive layer 240 and the piezoelectric film 250. In some embodiments, the adhesion-promoting layer 260 has a substantially similar material composition as the piezoelectric film 250. In these embodiments, the adhesion-promoting layer 260 may be formed along by mixing the piezoelectric polymer with the first and second solvents (e.g., MEK and DMA) according to the step 305 discussed above with reference to FIG. 4. As an alternative, either different solvents or different ratios may be employed to form a thin layer during the spin coating process.

In other embodiments, there are other alternatives to a PVDF-TrFE based adhesion-promoting layer 260. For example, alternative adhesion-promoting layers may include Chromium, a PBMA (poly n-butyl methacrylate) solution, or VM 652 (an adhesion promoter offered by 3M). It is also understood that a combination of all these materials discussed above to form the adhesion-promoting layer 260. For example, a layer of VM652 may be combined with an adhesion layer of PVDF-TrFE to form the adhesion-promoting layer 260.

Thereafter, the adhesion-promoting layer 260 is spin coated onto the surfaces of the dielectric layer 230 and the conductive layer 240. In certain embodiments, the adhesion-promoting layer 260 has a thickness in a range from about 0.3 um to about 0.7 um, for example about 0.5 um. The adhesion-promoting layer 260 is then baked on at a temperature of at least 110 degrees Celsius, for example between about 120 degrees Celsius and about 190 degrees Celsius. Thereafter, the piezoelectric film 250 is spin coated onto the adhesion-promoting layer 260 and processed in a manner similar to the steps 315-325 discussed above with reference to FIG. 4. As its name suggests, the adhesion-promoting layer 260 facilitates the adhesion of the piezoelectric film 250 to the dielectric layer 230 and the conductive layer 240 below. In other words, due to the presence of the adhesion-promoting layer 260, the piezoelectric film 250 is not easily peeled off, and that enhances the mechanical integrity of the transducer 200. It is understood that, in the illustrated embodiments, while the material compositions of the adhesion-promoting layer 260 and the piezoelectric film 250 may be substantially similar, they are two separate or discrete layers. In other words, a visible demarcation line or boundary exists between these two layers. This boundary can be observed under a microscope, for example. However, in alternative embodiments, it is also possible to melt or fuse these two layers together, so that they appear as a single layer.

In the embodiments shown in FIG. 3 and FIG. 5, after its spin coating deposition, the piezoelectric film 250 is patterned to achieve a desired shape, for example the shapes shown in FIGS. 3 and 5. Unwanted portions of the piezoelectric film 250 (and portions of the adhesion-promoting layer 260 therebelow) are removed in the patterning process. As a result, portions of the dielectric layer 230 and the conductive layer 240 are exposed.

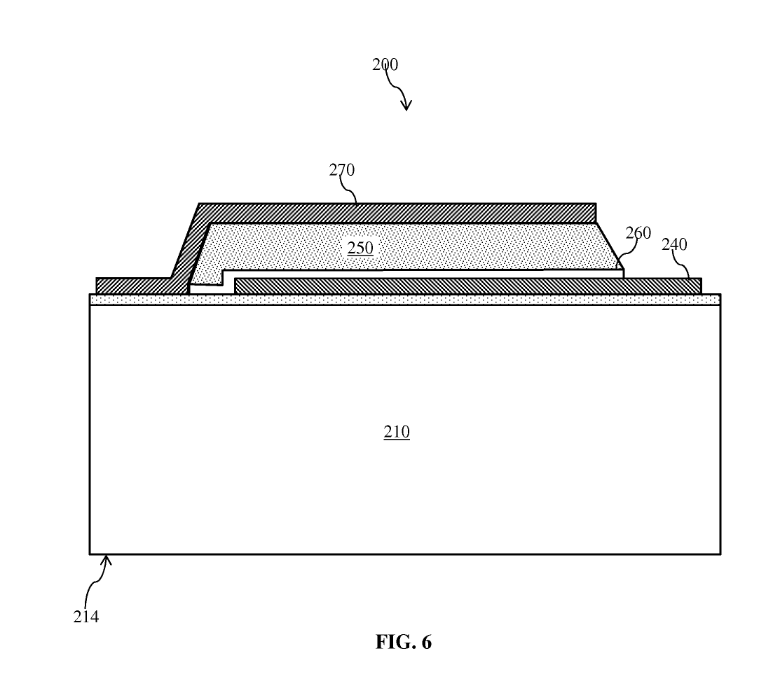

Referring now to FIG. 6, a conductive layer 270 is formed over the piezoelectric film 250 using a suitable deposition process known in the art. After its deposition, the conductive layer 270 is patterned using techniques in a photolithography process. Unwanted portions of the conductive layer 270 are removed as a part of the photolithography process. For reasons of simplicity, FIG. 6 only illustrates the conductive layer 270 after it has been patterned.

The conductive layers 240 and 270 and the piezoelectric layer 250 (and the adhesion-promoting layer 260 in embodiments where it is used) may collectively be considered a transducer membrane.

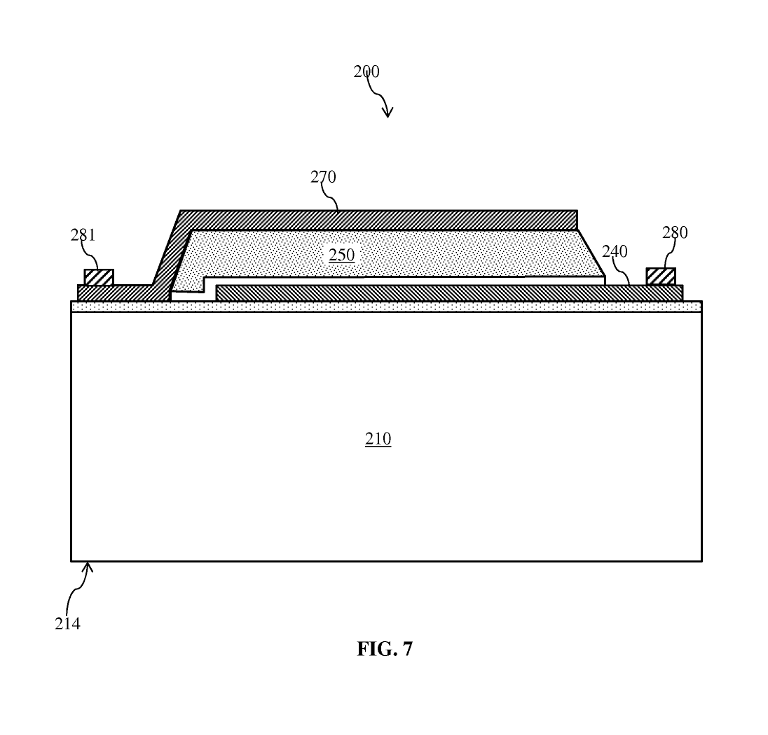

Referring now to FIG. 7, pad metals 280-281 are formed. The pad metal 280 is formed on, and electrically coupled, to the conductive layer 240, and the pad metal 281 is formed on and electrically coupled to the conductive layer 270. The pad metals 280-281 may be formed by depositing a layer of metal over the conductive layers 240 and 270 and thereafter patterning the layer of metal in a lithography process. As a result, the pad metals 280-281 are formed. The pad metals 280-281 may serve as electrodes for the transducer 200. Through these electrodes (i.e., the pad metals 280-281), electrical connections may be established between the transducer 200 and external devices such as electronic circuitry (not illustrated herein). The electronic circuitry can excite the transducer membrane so that it generates sound waves, particularly sound waves in an ultrasound range.

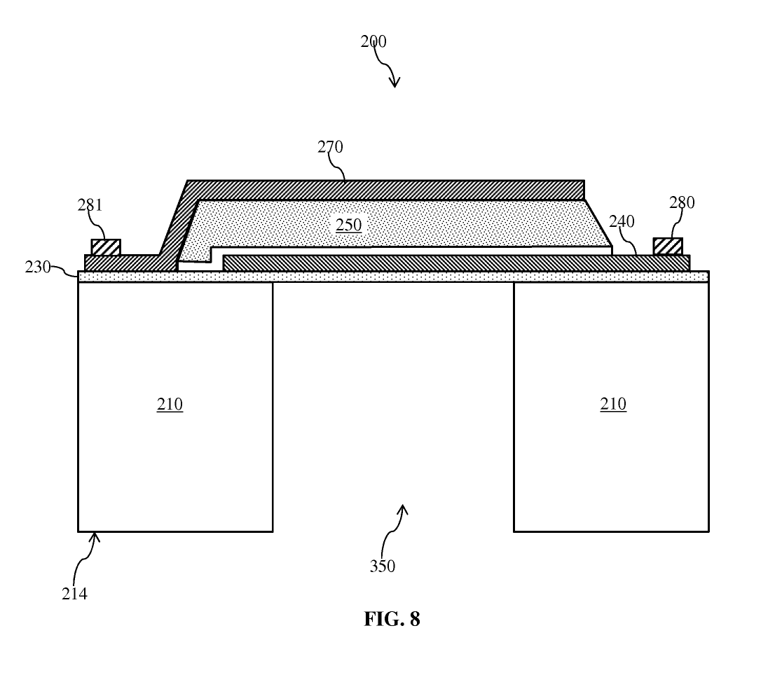

Referring now to FIG. 8, an opening 350 is formed in the substrate 210 from the back side 214. The opening 350 may also be referred to as a well, void, or a recess. The opening 350 is formed up to the dielectric layer 230. In other words, a portion of the dielectric layer 230 is exposed by the opening 350. In some embodiments, the opening 350 is formed by an etching process, for example a deep reactive ion etching (DRIE) process. The opening 350 forms an aperture of the transducer 200. Thereafter, the surface around the individual transducer 200 may be etched to define a singulated form factor for the device.

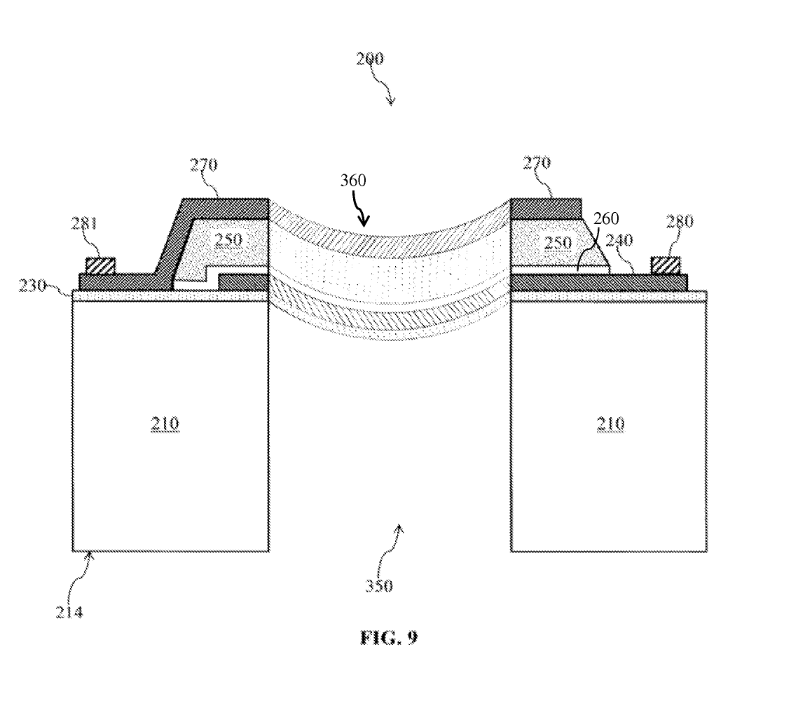

Referring now to FIG. 9, the opening 350 is deflected to form a concave surface. Stated differently, the portion of the dielectric layer 230 exposed by the opening 350 as well as the portions of the transducer membrane disposed over the portion of the dielectric layer 230 are bent toward the back side 214. Therefore, an arcuate-shaped transducer membrane 360 is formed. The arcuate shape of the transducer membrane 360 helps is spherically focus ultrasound signals emitted therefrom. In different embodiments, the transducer membrane 360 may exhibit other shaped configurations to achieve various other focusing characteristics. For example, in an alternative embodiment, the transducer membrane 360 may have a more arcuate shape or a more planar shape.

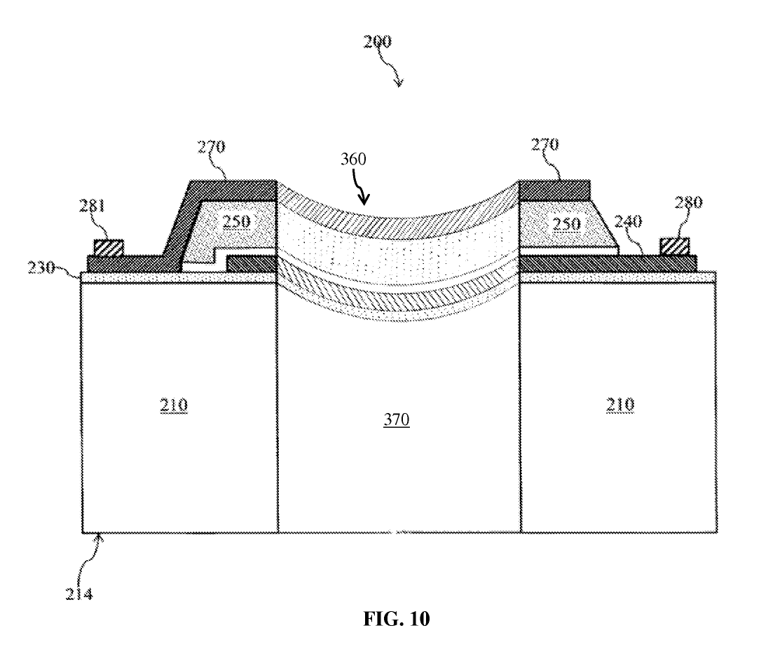

Referring now to FIG. 10, the opening 350 is filled with a backing material 370. The backing material 370 filling the opening 350 allows the aperture position to be fixed and also deadens the sound waves coming from the back of the piezoelectric film 250. In more detail, the backing material 370 physically contacts the bottom surface (or back side surface) of the dielectric layer 230. Therefore, one function of the backing material 370 is that it helps lock the transducer membrane 360 into place such that its shape (here, the arcuate shape) is maintained. The backing material 370 also contains an acoustically attenuative material so that it can absorb acoustic energy (in other words, sound waves) generated by the transducer membrane 360 that travels (propagates) into the ultrasound transducer 200 (for example, from the transducer membrane 360 into the backing material 370). Such acoustic energy includes acoustic energy that is reflected from structures and interfaces of a transducer assembly, for example when the ultrasound transducer 200 is included in the transducer assembly 122 of FIG. 1.

To adequately deaden the sound waves, the backing material 370 may have an acoustic impedance greater than about 4.5 megaRayls. In the present embodiment, the backing material 370 includes an epoxy material. In various other embodiments, the backing material 370 may include other materials that provide sufficient acoustical attenuation and mechanical strength for maintaining the shape of the transducer membrane 360. The backing material 370 may include a combination of materials for achieving such acoustical and mechanical properties. In some embodiments, the epoxy being used include EPO-Tek 301 or EPO-Tek 353ND. However, epoxy alone may not be sufficient as the backing material 370. In some embodiments, the epoxy is manipulated by adding filler materials such as Cerium Oxide or Tungsten Oxide. These materials are more dense. Density multiplied by the speed of sound equals acoustic impedance. For PVDF-TrFE transducers, a relatively high acoustic impedance is desired, and most if not all epoxies have low acoustic impedance. Therefore, filler materials are added to drive up the acoustic impedance and reflect sound that comes off the back of the transducer, back toward the front, which boosts the signal.

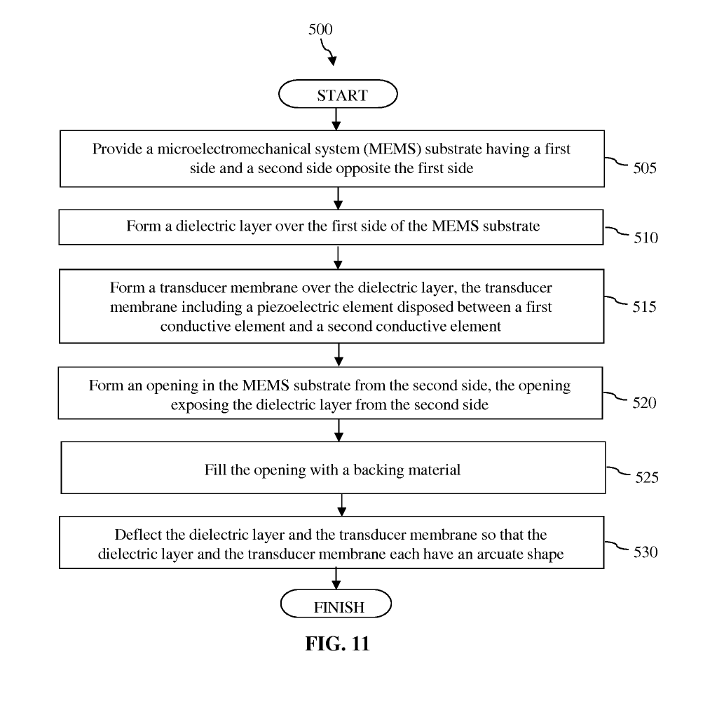

FIG. 11 is a flowchart of a method 500 for fabricating a polymeric MEMS-based ultrasonic transducer according to various aspects of the present disclosure. The method 500 includes a step 505, wherein a microelectromechanical system (MEMS) substrate is provided. The MEMS substrate has a first side and a second side opposite the first side. In some embodiments, the MEMS substrate is a silicon substrate and may contain microelectronic circuitry therein.

The method 500 includes a step 510, in which a dielectric layer is formed over the first side of the MEMS substrate. The dielectric layer may include silicon oxide, silicon nitride, silicon oxynitride, or combinations thereof. The dielectric layer provides a support surface for a multi-layered transducer membrane that is to be formed thereon.

The method 500 includes a step 515, in which the multi-layered transducer membrane is formed over the dielectric layer. The transducer membrane includes a piezoelectric element disposed between a first conductive element and a second conductive element. In some embodiments, the step 515 includes: depositing a first conductive layer over the dielectric layer; patterning the first conductive layer to form the first conductive element; spin casting a piezoelectric material over the first conductive element; annealing the piezoelectric material; etching the piezoelectric material to form the piezoelectric element; depositing a second conductive layer over the piezoelectric element; and patterning the second conductive layer to form the second conductive element. The way in which the piezoelectric material is spin cast over the first conductive element may be performed according to the method 300 shown in FIG. 4. The piezoelectric element may contain polyvinylidene fluoride (PVDF), polyvinylidene fluoride-trifluoroethylene (PVDF-TrFE), polyvinylidene fluoride-tetrafluoroethylene (PVDF-TFE), or combinations thereof.

The method 500 includes a step 520, in which the opening in the MEMS substrate is filled from the second side. The opening exposes the dielectric layer from the second side. The opening may be formed by an etching process such as a DRIE process.

The method 500 includes a step 525, in which the opening is filled with a backing material. The backing material contains an epoxy material. In some embodiments, the backing material has an acoustic impedance greater than about 4.5 megaRayls.

The method 500 includes a step 530, in which the dielectric layer and the transducer membrane are defected in a manner so that the dielectric layer and the transducer membrane each have an arcuate shape. The transducer membrane is conformally disposed on the dielectric layer. The arcuate shape of the transducer membrane allows the transducer membrane to focus sound beams. As a result, the transducer membrane (or the transducer itself) can operate at frequencies between 1 megahertz (MHz) and 135 MHz, for example in a frequency range from about 5 MHz to about 100 MHz.

It is understood that additional fabrication steps may be performed to complete the fabrication of the transducer. However, these additional fabrication steps are not discussed herein for reasons of simplicity.

The polymeric MEMS-based transducer manufactured according to the present disclosure can perform imaging tasks with ultrasound with less than about a 50 um resolution. In addition, the polymeric MEMS-based transducer of the present disclosure can achieve about a 10 millimeter (mm) depth of penetration.

One aspect of the present disclosure involves a method of fabricating an ultrasound transducer. The method includes: mixing a piezoelectric polymer into a solution containing a first chemical and a second chemical to form a viscous film; coating the film onto a wafer, wherein the first chemical is substantially flashed off during the coating; thereafter baking the film, wherein the second chemical is substantially removed during the baking; and thereafter annealing the film, wherein the film has a β phase crystallinity greater than 50% after the annealing.

In some embodiments, the method further includes, before the coating: applying an adhesion-promoting layer over the wafer in a baking process, wherein the adhesion-promoting layer is substantially thinner than the film, and wherein the film is coated on the adhesion-promoting layer.

In some embodiments, the adhesion-promoting layer has a substantially similar material composition as the film.

In some embodiments, the adhesion-promoting layer has a thickness in a range from about 0.3 microns to about 0.7 microns.

In some embodiments, the coating the film is performed using a spin-coating process.

In some embodiments, the film is a part of a multi-layered transducer membrane, and further comprising: deflecting the transducer membrane so that the transducer membrane has a concave shape.

In some embodiments, the first chemical includes methyl ethyl ketone (MEK); and the second chemical includes dimethylacetamide (DMA).

In some embodiments, the first chemical includes cyclohexanone; and the second chemical includes dimethyl sulfoxide (DMSO).

In some embodiments, the piezoelectric polymer contains polyvinylidene fluoride-trifluoroethylene (PVDF-TrFE), polyvinylidene fluoride (PVDF), or polyvinylidene fluoride-tetrafluoroethylene (PVDF-TFE).

In some embodiments, the piezoelectric polymer, the first chemical, and the second chemical have a mixing ratio by weight of about (2˜3):(6˜8):(2˜4). In some embodiments, the mixing ratio is about (2.5˜2.8):(6.5˜7.5):(2.5˜3.5). In some embodiments, the mixing ratio is about 2.66:7:3.

In some embodiments, the film has a thickness in a range from about 8 microns to about 10 microns.

In some embodiments, the film has a viscosity in a range from about 575 centipoise (cP) to about 625 cP.

In some embodiments, the coating is performed such that a significant portion of the second chemical remains after the coating.

In some embodiments, the annealing is performed using an annealing temperature in a range from about 135 degrees Celsius to about 145 degrees Celsius and an annealing duration in a range from about 17 hours to about 19 hours.

Another aspect of the present disclosure involves a micromachined ultrasound transducer. The micromachined ultrasound transducer includes: a substrate; an opening formed in the substrate, the opening being filled with a backing material; a first metal layer disposed over the backing material; an adhesion-promoting layer disposed over the first metal layer; a piezoelectric layer disposed over the adhesion-promoting layer, the piezoelectric layer being substantially thicker than the adhesion-promoting layer; and a second metal layer disposed over the piezoelectric layer; wherein the first metal layer, the adhesion-promoting layer, the piezoelectric layer, and the second metal layer are each a part of a transducer membrane of the micromachined ultrasonic transducer.

In some embodiments, the backing material has a concave surface over which the first metal layer is disposed.

In some embodiments, the first metal layer is conformally disposed over the backing material; the adhesion-promoting layer is conformally disposed over the first metal layer; the piezoelectric layer is disposed over the adhesion-promoting layer; and the second metal layer is disposed over the piezoelectric layer.

In some embodiments, the adhesion-promoting layer has a thickness is a range from about 0.3 microns to about 0.7 microns; and the piezoelectric layer has a thickness is a range from about 8 microns to about 10 microns.

In some embodiments, the adhesion-promoting layer and the piezoelectric layer have substantially similar material compositions.

In some embodiments, the piezoelectric layer contains polyvinylidene fluoride (PVDF), polyvinylidene fluoride-trifluoroethylene (PVDF-TrFE), or polyvinylidene fluoride-tetrafluoroethylene (PVDF-TFE).

In some embodiments, the piezoelectric layer has a β phase crystallinity greater than 60%.

Yet another aspect of the present disclosure involves an ultrasound system. The ultrasound system includes: an imaging component that includes a flexible elongate member and a piezoelectric micromachined ultrasound transducer (PMUT) coupled to a distal end of the elongate member, wherein the PMUT includes: a substrate having a front surface and a back surface opposite the first surface; a well located in the substrate, the well extending from the back surface of the substrate to, but not beyond, the front surface of the substrate; a first metal layer disposed over the well, wherein a segment of the first metal layer disposed over the well has an arcuate shape; an adhesion-promoting film disposed over the first metal layer; a piezoelectric film disposed over the adhesion-promoting film, the piezoelectric film being substantially thicker than the adhesion-promoting film; and a second metal layer disposed over the piezoelectric film; an interface module configured to engage with a proximal end of the elongate member; and an ultrasound processing component in communication with the interface module.

In some embodiments, the adhesion-promoting film has a thickness is a range from about 0.3 microns to about 0.7 microns; and the piezoelectric film has a thickness is a range from about 8 microns to about 10 microns.

In some embodiments, the adhesion-promoting film and the piezoelectric film have substantially similar material compositions.

In some embodiments, the piezoelectric film has a β phase crystallinity greater than 60%.

In some embodiments, the well is filled by a backing material configured to absorb energy transmitted by the piezoelectric film. In some embodiments, the backing material contains epoxy.

In some embodiments, the piezoelectric film is configured to operate at frequencies between 1 megahertz (MHz) and 135 MHz.

In some embodiments, the piezoelectric film contains polyvinylidene fluoride (PVDF), polyvinylidene fluoride-trifluoroethylene (PVDF-TrFE), or polyvinylidene fluoride-tetrafluoroethylene (PVDF-TFE).

Another aspect of the present disclosure involves a micromachined ultrasound transducer. The micromachined ultrasound transducer includes: a substrate having a first side and a second side opposite the first side; a well disposed in the substrate; an insulating film disposed over the well and over the substrate on the first side, the insulating film having a concave surface facing the first side; a first conductive layer disposed over a portion of the insulating film on the first side; a piezoelectric element disposed over the first conductive layer on the first side; and a second conductive layer disposed over the piezoelectric element on the first side.

In some embodiments, portions of the first and second conductive layers and the piezoelectric element disposed over the well each have a curved shape.

In some embodiments, the well is located entirely within the substrate and is filled by a backing material. In some embodiments, the backing material has an acoustic impedance greater than about 4.5 megaRayls. In some embodiments, the insulating film contains a dielectric material; and the backing material contains an epoxy material.

In some embodiments, the piezoelectric element is configured to operate at frequencies between 1 megahertz (MHz) and 135 MHz.

In some embodiments, the piezoelectric element contains polyvinylidene fluoride (PVDF), polyvinylidene fluoride-trifluoroethylene (PVDF-TrFE), or polyvinylidene fluoride-tetrafluoroethylene (PVDF-TFE).

In some embodiments, the substrate is a microelectromechanical system (MEMS) substrate.

Another aspect of the present disclosure involves an ultrasound system. The ultrasound system includes: an imaging component that includes a flexible elongate member and a piezoelectric micromachined ultrasound transducer (PMUT) coupled to a distal end of the elongate member, wherein the PMUT includes: a substrate having a front surface and a back surface opposite the first surface; a well located in the substrate, the well extending from the back surface of the substrate to, but not beyond, the front surface of the substrate; a dielectric support layer disposed over the well and over the front surface of the substrate, wherein a portion of the dielectric support layer disposed over the well has an arcuate shape; and a transducer membrane disposed conformally over the dielectric support layer, wherein the transducer member includes a piezoelectric element disposed between a first conductive element and a second conductive element; an interface module configured to engage with a proximal end of the elongate member; and an ultrasound processing component in communication with the interface module.

In some embodiments, the well is filled by a backing material configured to absorb energy transmitted by the piezoelectric element. In some embodiments, the backing material contains epoxy.

In some embodiments, the piezoelectric element is configured to operate at frequencies between 1 megahertz (MHz) and 135 MHz.

In some embodiments, the piezoelectric element contains polyvinylidene fluoride (PVDF), polyvinylidene fluoride-trifluoroethylene (PVDF-TrFE), or polyvinylidene fluoride-tetrafluoroethylene (PVDF-TFE).

Another aspect of the present disclosure involves a method of fabricating an ultrasound transducer. The method includes: providing a substrate having a first side and a second side opposite the first side; forming a dielectric layer over the first side of the substrate; forming a transducer membrane over the dielectric layer, the transducer membrane including a piezoelectric element disposed between a first conductive element and a second conductive element; forming an opening in the substrate from the second side, the opening exposing the dielectric layer from the second side; and deflecting the dielectric layer and the transducer membrane so that the dielectric layer and the transducer membrane each have an arcuate shape.

In some embodiments, the forming the transducer membrane comprises: depositing a first conductive layer over the dielectric layer; patterning the first conductive layer to form the first conductive element; spin casting a piezoelectric material over the first conductive element; annealing the piezoelectric material; etching the piezoelectric material to form the piezoelectric element; depositing a second conductive layer over the piezoelectric element; and patterning the second conductive layer to form the second conductive element.

In some embodiments, the method further includes: filling the opening with a backing material.

In some embodiments, the backing material has an acoustic impedance greater than about 4.5 megaRayls. In some embodiments, the backing material contains an epoxy material. In some embodiments, the transducer membrane is configured to operate at frequencies between 1 megahertz (MHz) and 135 MHz.

In some embodiments, the piezoelectric element contains polyvinylidene fluoride (PVDF), polyvinylidene fluoride-trifluoroethylene (PVDF-TrFE), or polyvinylidene fluoride-tetrafluoroethylene (PVDF-TFE).

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A method of fabricating an ultrasound transducer, the method comprising:
mixing a piezoelectric polymer into a solution containing a first chemical and a second chemical to form a viscous film;
coating the film onto a wafer, wherein the first chemical is substantially flashed off during the coating;
thereafter baking the film, wherein the second chemical is substantially removed during the baking; and
thereafter annealing the film, wherein the film has a β phase crystallinity greater than 50% after the annealing.

2. The method of claim 1, further comprising, before the coating: applying an adhesion-promoting layer over the wafer in a baking process, wherein the adhesion-promoting layer is substantially thinner than the film, and wherein the film is coated on the adhesion-promoting layer.

3. The method of claim 2, wherein the adhesion-promoting layer has a substantially similar material composition as the film and has a thickness in a range from about 0.3 microns to about 0.7 microns.

4. The method of claim 1, wherein the coating the film is performed using a spin-coating process.

5. The method of claim 1, wherein the film is a part of a multi-layered transducer membrane, and further comprising: deflecting the transducer membrane so that the transducer membrane has a concave shape.

6. The method of claim 1, wherein:
the first chemical includes methyl ethyl ketone (MEK); and
the second chemical includes dimethylacetamide (DMA).

7. The method of claim 1, wherein:
the first chemical includes cyclohexanone; and
the second chemical includes dimethyl sulfoxide (DMSO).

8. The method of claim 1, wherein the piezoelectric polymer contains polyvinylidene fluoride-trifluoroethylene (PVDF-TrFE), polyvinylidene fluoride (PVDF), or polyvinylidene fluoride-tetrafluoroethylene (PVDF-TFE).

9. The method of claim 1, wherein: the piezoelectric polymer, the first chemical, and the second chemical have a mixing ratio by weight of about (2˜3):(6˜8):(2˜4).

10. The method of claim 9, wherein the mixing ratio is about (2.5˜2.8):(6.5˜7.5):(2.5˜3.5).

11. The method of claim 1, wherein:
the film has a thickness in a range from about 8 microns to about 10 microns; and
the film has a viscosity in a range from about 575 centipoise (cP) to about 625 cP.

12. The method of claim 1, wherein the coating is performed such that a significant portion of the second chemical remains after the coating.

13. The method of claim 1, wherein the annealing is performed using an annealing temperature in a range from about 135 degrees Celsius to about 145 degrees Celsius and an annealing duration in a range from about 17 hours to about 19 hours.

14. A micromachined ultrasound transducer, comprising:
a substrate;
an opening formed in the substrate, the opening being filled with a backing material;
a first metal layer disposed over the backing material;
an adhesion-promoting layer disposed over the first metal layer;
a piezoelectric layer disposed over the adhesion-promoting layer, the piezoelectric layer being substantially thicker than the adhesion-promoting layer; and
a second metal layer disposed over the piezoelectric layer;
wherein the first metal layer, the adhesion-promoting layer, the piezoelectric layer, and the second metal layer are each a part of a transducer membrane of the micromachined ultrasonic transducer.

15. The micromachined ultrasound transducer of claim 14, wherein the backing material has a concave surface over which the first metal layer is disposed.

16. The micromachined ultrasound transducer of claim 14, wherein:
the first metal layer is conformally disposed over the backing material;
the adhesion-promoting layer is conformally disposed over the first metal layer, wherein the adhesion-promoting layer has a thickness is a range from about 0.3 microns to about 0.7 microns;
the piezoelectric layer is disposed over the adhesion-promoting layer, wherein the piezoelectric layer has a thickness is a range from about 8 microns to about 10 microns; and
the second metal layer is disposed over the piezoelectric layer.

17. The micromachined ultrasound transducer of claim 14, wherein the adhesion-promoting layer and the piezoelectric layer have substantially similar material compositions.

18. The micromachined ultrasound transducer of claim 14, wherein the piezoelectric layer contains polyvinylidene fluoride (PVDF), polyvinylidene fluoride-trifluoroethylene (PVDF-TrFE), or polyvinylidene fluoride-tetrafluoroethylene (PVDF-TFE).

19. The micromachined ultrasound transducer of claim 14, wherein the piezoelectric layer has a β phase crystallinity greater than 60%.

20. An ultrasound system, comprising:
an imaging component that includes a flexible elongate member and a piezoelectric micromachined ultrasound transducer (PMUT) coupled to a distal end of the elongate member, wherein the PMUT includes:
a substrate having a front surface and a back surface opposite the first surface;
a well located in the substrate, the well extending from the back surface of the substrate to, but not beyond, the front surface of the substrate, wherein the well is filled by a backing material configured to absorb energy transmitted by the piezoelectric film;
a first metal layer disposed over the well, wherein a segment of the first metal layer disposed over the well has an arcuate shape;
an adhesion-promoting film disposed over the first metal layer;
a piezoelectric film disposed over the adhesion-promoting film, the piezoelectric film being substantially thicker than the adhesion-promoting film, wherein the piezoelectric film has a β phase crystallinity greater than 60%, and wherein the piezoelectric film contains polyvinylidene fluoride (PVDF), polyvinylidene fluoride-trifluoroethylene (PVDF-TrFE), or polyvinylidene fluoride-tetrafluoroethylene (PVDF-TFE); and
a second metal layer disposed over the piezoelectric film;
an interface module configured to engage with a proximal end of the elongate member; and
an ultrasound processing component in communication with the interface module.

21. The ultrasound system of claim 20, wherein:
the adhesion-promoting film has a thickness is a range from about 0.3 microns to about 0.7 microns; and
the piezoelectric film has a thickness is a range from about 8 microns to about 10 microns.

22. The ultrasound system of claim 20, wherein the adhesion-promoting film and the piezoelectric film have substantially similar material compositions.

23. The ultrasound system of claim 20, wherein the backing material contains epoxy and a filler material.

24. The ultrasound system of claim 20, wherein the piezoelectric film is configured to operate at frequencies between 1 megahertz (MHz) and 135 MHz.
